(19) United States
(12) Patent Application Publication
(10) Pub. No.: US 2003/0139430 A1
(43) Pub. Date: Jul. 24, 2003

(54) Use of organic compounds

(76) Inventors: Debra Jane Resta, Somerset, NJ (US); Kurt Clement Sizer, Hoboken, NJ (US); Peter Traxler, Schonenbuch (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/348,716

(22) Filed: Jan. 22, 2003

Related U.S. Application Data
(60) Provisional application No. 60/351,591, filed on Jan. 24, 2002.

(51) Int. Cl. A61K 31/519

(52) U.S. Cl. 514/265.1

(57) ABSTRACT

This disclosure relates to the treatment of cancer, particularly breast cancer, with a combination an aromatase inhibitor, such as letrozole, and a compound that inhibits the tyrosine kinase activity of epidermal growth factor (EGF). Methods of treatment and pharmaceutical compositions are included in the disclosure.

The invention relates to a pharmaceutical combination which comprises (a) letrozole and (b) a 7H-pyrrolo[2,3-d]pyrimidine derivative of formula I (see below) and optionally at least one pharmaceutically acceptable carrier for simultaneous, separate or sequential use, in particular for the treatment of a solid tumor disease; a pharmaceutical composition comprising such a combination; the use of such a combination for the preparation of a medicament for the treatment of a solid tumor disease; a commercial package or product comprising such a combination as a combined preparation for simultaneous, separate or sequential use; and to a method of treatment of a warm-blooded animal, especially a human.

Letrozole inhibits in humans the estrogen production, i.e. the conversion of the substrates androstenedione and testosterone to estrone and estradiol, respectively. The compound is particularly useful for the treatment of hormone receptor positive breast tumors.

Compounds which inhibit the tyrosine kinase activity of the epidermal growth factor (EGF) receptor are useful, for example, in the treatment of benign or malignant tumours. They are capable of preventing the formation of tumour metastases and the growth of micro-metastases. They can be used especially in the case of epidermal hyperproliferation (psoriasis), in the treatment of neoplasias of epithelial character and in leukemias. The 7H-pyrrolo[2,3-d]pyrimidines disclosed in WO 97/02266 represent inhibitors of the EGF receptor tyrosine kinase activity.

Surprisingly, it has now been found that the anti-proliferative effect of a combination which comprises letrozole and 7H-pyrrolo[2,3-d]pyrimidine derivatives of formula I

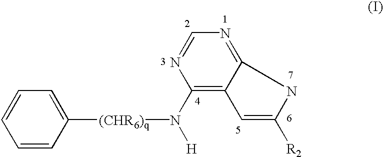

wherein q is 1, R₂ is phenyl substituted by hydroxy, and R₆ is hydrogen or methyl; is greater than the maximum effect that can be achieved with either type of ingredient alone.

Hence, the present invention pertains to a combination, such as a combined preparation or a pharmaceutical composition, which comprises (a) letrozole and (b) a 7H-pyrrolo[2,3-d]pyrimidine derivative of formula I as defined above in which the active ingredients (a) and (b) are present in each case in free form or in the form of a pharmaceutically acceptable salt and optionally at least one pharmaceutically acceptable carrier; for simultaneous, separate or sequential use, in particular for the treatment of a solid tumor disease.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutical effect in a non-effective dosage of one or both of the combination partners (a) and (b), and very preferably a strong synergism of the combination partners (a) and (b).

The term “treatment” comprises the administration of the combination partners to a warm-blooded animal in need of, such treatment with the aim to effect a delay of progression of a disease. The term delay of “progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

The term “a solid tumor disease” especially means breast cancer, ovarian cancer, cancer of the colon and generally the GI tract, cervix cancer, lung cancer, e.g. small-cell lung cancer and non-small-cell lung cancer, head and neck cancer, renal cancer, bladder cancer, cancer of the prostate, glioma or Kaposi's sarcoma.

Letrozole can be prepared as described in U.S. Pat. No. 5,473,078. It can be administered, e.g., as described in U.S. Pat. No. 4,978,672 or U.S. Pat. No. 5,473,078, or in the form as it is marketed, e.g. under the trademark FEMARA™ or FEMAR™.

The 7H-pyrrolo[2,3-d]pyrimidines of formula I can be prepared and administered as disclosed in WO 97/02266. Preferably, the compounds of formula I are administered orally, e.g., in hard gelatin capsules.

The compounds used as combination partners (a) and (b) disclosed herein can be prepared and administered as described in the cited documents, respectively. The combination partners (a) or (b) and their salts may also be used in the form of a hydrate or include other solvents used for crystallization.

A combination which comprises (a) letrozole and (b) a compound of formula I wherein q is 1, R₂ is phenyl substituted by hydroxy, and R₆ is hydrogen or methyl, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATIONS OF THE INVENTION inhibits the growth of solid tumors. In one preferred embodiment of the invention, the solid tumor disease to be treated with a COMBINATION OF THE INVENTION is breast cancer, in particular advanced breast cancer in post-menopausal women, and especially breast cancer wherein the tumors are either estrogen-receptor and/or progesterone-receptor positive and/or positive for type 1 growth factors (EGFR/HER2). Furthermore, the COMBINATIONS OF THE INVENTION is in particular suitable for the treatment of breast cancer which is at least partially resistant to the treatment with tamoxifen.

The nature of solid tumor diseases is multifactorial. Under certain circumstances, drugs with different mechanisms of action may be combined. However, just considering any combination of drugs having different mode of action does not necessarily lead to combinations with advantageous effects.

All the more surprising is the experimental finding that in vivo the administration of a COMBINATION OF THE INVENTION compared to a monotherapy applying only one of the pharmaceutically active ingredients used in the COMBINATION OF THE INVENTION results not only in a more beneficial, especially synergistic, e.g. anti-proliferative effect, e.g. with regard to the delay of progression of a proliferative disease or with regard to a change in tumor volume, but also in further surprising beneficial effects, e.g. less side-effects and a decreased mortality and morbidity. Furthermore, depending on the particular tumor type a decrease of the tumor volume can be obtained when using a COMBINATION OF THE INVENTION in cases in which by monotherapy no decrease of the tumor volume can be achieved. The COMBINATIONS OF THE INVENTION are also suitable to prevent the metastatic spread of tumors and the growth or development of micrometastases. The COMBINATIONS OF THE INVENTION are in particular suitable for the treatment of patients with advanced cancer who have failed standard systemic therapy. This includes patients having tumor types showing resistance to monotherapy, especially monotherapy with tamoxifen, or showing resistance to combinations different from those disclosed herein.

A further benefit is that lower doses of the active ingredients of the COMBINATION OF THE INVENTION can be used, for example, that the dosages need not only often be smaller, but are also applied less frequently, or can be used in order to diminish the incidence of side-effects observed with one of the combination partners alone. This is in accordance with the desires and requirements of the patients to be treated.

It can be shown by established test models that a COMBINATION OF THE INVENTION results in the beneficial effects described herein-before. The person skilled in the pertinent art is fully enabled to select a relevant test model to prove such beneficial effects. The pharmacological activity of a COMBINATION OF THE INVENTION may, for example, be demonstrated in a clinical study or in a test procedure as essentially described hereinafter.

Suitable clinical studies are, e.g., randomized, double-blind, placebo-controlled, parallel studies in female breast cancer patients with advanced disease having tumors which are either estrogen-receptor and/or progesterone-receptor positive. Such studies are, in particular, suitable to compare the effects of amonotherapy using the active ingredients and a therapy using a COMBINATION OF THE INVENTION, and to prove in particular the synergism of the active ingredients of the COMBINATIONS OF THE INVENTION. The primary endpoints in such studies can be the effect on pain scores, analgesic use, performance status, Quality of Life scores, time to progression of the disease, morbidity or mortality. The radiologic evaluation of tumors in regular time periods, e.g. every 8 or 12 weeks, is a suitable approach to determine the effect of the COMBINATION OF THE INVENTION. In a suitable study design, patients are, for example, randomized in a double-blind fashion receiving letrozole in a daily dose of 2.5 mg in addition to a daily dose of 200, 400, 600 or 800 mg PKI166 or a corresponding placebo. The minimum duration of such a study should be about 6 or 12 months.

It is one objective of this invention to provide a pharmaceutical composition comprising a quantity, which is jointly therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man, comprising a thera-peutically effective amount of at least one pharmacologically active combination partner alone or in combination with one or more pharmaceutically acceptable carries, especially suitable for enteral or parenteral application. In one embodiment of the invention, one or more of the active ingredients are administered intraveniously.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partners of the COMBINATION OF THE INVENTION may be administered simultaneously or sequentially and in any order, and the components may be administered separately or as a fixed combination. For example, the method of treatment of a solid tumor disease according to the invention may comprise (i) administration of the first combination partner in free or pharmaceutically acceptable salt form and (ii) adminstration of the second combination partner in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. This involves a consideration of the distribution, equilibrium, and elimination of the active ingredients.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

In particular, if the the warm-blooded animal is a human, the dosage of a compound of formula I is preferably in the range of about is in the range from about 50 mg to about 2000 mg/day.

Letrozole is preferably administered daily according to the package insert at a dose of 2.5 mg.

Preferably, a compound of formula I is employed wherein q is 1, n is 0, R₁ is hydrogen, R₂ is phenyl substituted by 4-hydroxy, and R₆ is methyl. This particular compound is also known as “PKI166”.

The COMBINATION OF THE INVENTION can be a combined preparation or a pharmaceutical composition.

Moreover, the present invention relates to a method of treating a warm-blooded animal having a solid tumor disease comprising administering to the animal a COMBINATION OF THE INVENTION in a quantity which is jointly therapeutically effective against a solid tumor disease and in which the combination partners can also be present in the form of their pharmaceutically acceptable salts. Furthermore, the treatment can comprise surgery, radiotherapy, cryotherapy and immunotherapy.

The present invention also provides a method of inhibiting the formation of metastases in a warm-blooded animal having a breast tumor disease which comprises administering to the patient a pharmaceutically effective amount of a COMBINATION OF THE INVENTION in a quantity which is jointly therapeutically effective against said tumor disease and in which the compounds can also be present in the form of their pharmaceutically acceptable salts.

In one embodiment of the invention, a compound of formula I is administered to a human subject less frequently than on a daily basis. In particular, such embodiment relates to a treatment regimen whereby over at least a three week period, a compound of formula I is administered on only about 40% to about 71% of the days. In such embodiment, specifically, the present invention relates to a method of treating a human subject with a compound of formula I, which comprises administering such pyrimidine derivative to the human subject from three to five times in each seven day period for a period of three weeks or longer, more specifically, three or four times a week on alternate days for a period of three weeks or longer. In a more specific embodiment, a compound of formula I is administered three times each week on alternate days, for example, on Monday, Wednesday and Friday of each week, for at least three weeks. Preferably, such dosage regimen is carried out through at least four or more weeks, for example 4, 5, 6, 7 or 8 weeks.

Alternatively, a compound of formula I is administered daily in cycles comprising a period of one to four weeks, e.g. two weeks, which is optionally followed by a period of one to three weeks, e.g. two weeks, without administering the compound to the patient. The whole treatment period consisting of such cycles can amount, e.g., to about 6, 12 or 18 months. Furthermore, the present invention pertains to the use of a COMBINATION OF THE INVENTION for the treatment of a solid tumor disease and for the preparation of a medicament for the treatment of a solid tumor disease.

Furthermore, the present invention pertains to the use of a COMBINATION OF THE INVENTION for the treatment of a solid tumor disease and for the preparation of a medicament for the treatment of a solid tumor disease.

Additionally, the present invention pertains to the use of letrozole in combination with a 7H-pyrrolo[2,3-d]pyrimidine derivative of formula I wherein q is 1, R₂ is phenyl substituted by hydroxy, and R₆ is hydrogen or methyl, for the preparation of a medicament for the treatment of a solid tumor disease.

Moreover, the present invention provides a commercial package comprising as active ingredients COMBINATION OF THE INVENTION, together with instructions for simultaneous, separate or sequential use thereof in the treatment of a solid tumor disease.

What is claimed is:

1. A combination which comprises (a) letrozole and (b) a 7H-pyrrolo[2,3-d]pyrimidine derivative of formula I 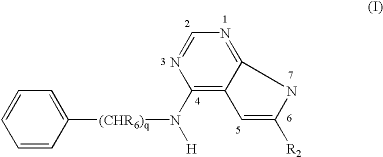
wherein q is 1, R₂ is phenyl substituted by hydroxy, and R₆ is hydrogen or methyl;
in which the active ingredients (a) and (b) are present in each case in free form or in the form of a pharmaceutically acceptable salt and optionally at least one pharmaceutically acceptable carrier; for simultaneous, separate or sequential use.

2. Combination according to claim 1 comprising a 7H-pyrrolo[2,3-d]pyrimidine derivative of formula I wherein q is 1, R₂ is phenyl substituted by 4-hydroxy, and R₆ is methyl.

3. Combination according to claim 1 or 2 which is a combined preparation or a pharmaceutical composition.

4. Method of treating a warm-blooded animal having a solid tumor disease which comprises administering to the animal a combination according to any one of claims 1 to 3 in a quantity which is jointly therapeutically effective against said tumor disease and in which the compounds can also be present in the form of their pharmaceutically acceptable salts.

5. Method of inhibiting the formation of metastases in a warm-blooded animal having a breast tumor disease which comprises administering to the patient a pharmaceutically effective amount of a combination according to any one of claims 1 to 3 in a quantity which is jointly therapeutically effective against said tumor disease and in which the compounds can also be present in the form of their pharmaceutically acceptable salts.

6. Method according to claim 4 or 5 which comprises administering the 7H-pyrrolo[2,3-d]pyrimidine derivative of formula I, or a salt thereof, to the human subject over at least a three week time period on only about 40% to about 71% of the days in the time period.

7. Method of claim 4 or 5 wherein the pharmaceutically effective daily dose of the 7H-pyrrolo[2,3-d]pyrimidine derivative of formula 1, or a salt thereof, is in the range from about 50 mg to about 2000 mg.

8. A pharmaceutical composition comprising a quantity which is jointly therapeutically effective against a solid tumor disease of a pharmaceutical combination according to any one of claims 1 to 3 and at least one pharmaceutically acceptable carrier.

9. Use of letrozole in combination with a 7H-pyrrolo[2,3-d]pyrimidine derivative of formula I 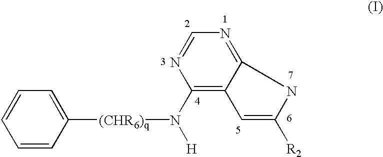
wherein q is 1, R₂ is phenyl substituted by hydroxy, and R₆ is hydrogen or methyl, for the preparation of a medicament for the treatment of a solid tumor disease.

10. A commercial package comprising (a) letrozole and (b) a 7H-pyrrolo[2,3-d]pyrimidine derivative of formula I 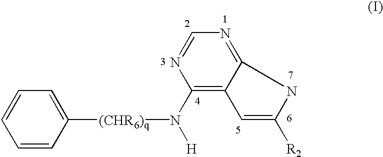
wherein q is 1, R₂ is phenyl substituted by hydroxy, and R₆ is hydrogen or methyl;
together with instructions for simultaneous, separate or sequential use thereof in the treatment of a solid tumor disease.
